(19) United States
(12) Patent Application Publication
(10) Pub. No.: US 2015/0027953 A1
(43) Pub. Date: Jan. 29, 2015

(54) APPLICATION OF MACROPOROUS SILICA SYNTHESIZED BY A SALT-TEMPLATED AEROSOL METHOD FOR CHROMATOGRAPHY

(71) Applicant: INDIANA UNIVERSITY RESEARCH AND TECHNOLOGY CORPORATION, Indianapolis, IN (US)

(72) Inventors: Amanda K. Peterson Mann, Bloomington, IN (US); Benjamin F. Mann, Bloomington, IN (US); Sara E. Skrabalak, Bloomington, IN (US); Milos V. Novotny, Bloomington, IN (US)

(21) Appl. No.: 14/371,193

(22) PCT Filed: Jan. 9, 2013
(86) PCT No.: PCT/US2013/020768

§ 371 Date: Jul. 9, 2014

Related U.S. Application Data
(60) Provisional application No. 61/585,445, filed on Jan. 11, 2012.

(51) Int. Cl. B01J 20/286; B01D 15/38; B01J 20/283; B01D 15/08

(52) U.S. Cl. CPC B01J 20/286; B01D 15/08; B01D 15/3804; B01J 20/283; B01J 2220/80
USPC 210/656; 502/407; 502/403

(57) ABSTRACT

The present invention discloses a silica particle having a diameter less than or equal to 2 μη, wherein the particle is spherical and comprises interconnected pores having a diameter in the range from 50 nm to 300 nm. The silica particle is preferably produced by spray pyrolysis (=spray drying) of a silica colloid. In the production process, porosity is introduced by means of an inorganic salt, such as NaCl, KCI, LiCl, NaNO3 or Ll NO3, which serves as a pore template. The silica particle may further be functionalized with proteins, peptides, nucleic acids, polysaccharides and proteoglycans, preferably concanavalin A or avidin. The present invention further discloses the use of the silica particle in chromatography, in particular in affinity chromatography.

### CROSS-REFERENCE TO RELATED APPLICATIONS

This application claims priority under 35 U.S.C. §119(e) to U.S. Provisional Application Ser. No. 61/585,445, filed Jan. 11, 2012, which is expressly incorporated by reference herein.

### TECHNICAL FIELD

The present invention relates generally to stationary support material and uses thereof in chromatography. More particularly, the present invention relates to macroporous silica and its use as a stationary phase in liquid chromatography.

### BACKGROUND

In liquid chromatography, the stationary phase support material used to pack the chromatography column plays a fundamental role in the separation process. One of the most common support materials is microparticulate silicon dioxide (hereinafter “SiO₂” or “silica”). SiO₂ is a well-suited material for use as a stationary phase support because it is rigid, chemically inert, and stable at high pressures. In high-pressure and ultra-high pressure liquid chromatography (HPLC and UPLC, respectively), the support material must be able to withstand pressure drops of several thousands to several tens of thousands of pounds per square inch. High back pressures are generated because it is desirable to use small particles that provide a high surface area per unit of column length, thereby allowing for a large number of interactions between molecules in the flowing liquid (i.e., the mobile phase) and the biological and/or chemical moieties bound to the support material (i.e., the stationary phase). Additionally, surface area can be increased by utilizing particles that are porous rather than solid. Enhancing porosity is an attractive approach to augment the surface area of a support material because it does not significantly increase the pressure drop across the chromatography column. Currently, completely solid particles, particles that are porous throughout, and particles that have a porous outer layer with a solid inner core are commercially available as chromatography support materials. While there are a wide variety of silica particles currently available, the processes by which they are synthesized impose limitations on the architectures that may be generated.

Two general synthetic routes are employed to generate porous silica particles, namely sol-gel polymerization and aerosol processes. Sol-gel polymerization is a process by which an organometallic solution (sol) undergoes hydrolysis to form a 3-D gel network (gel), followed by drying to produce a rigid network (i.e., the vacancies in the gel formed upon solvent loss (drying) result in pores). The nature of the precursor, basicity/acidity, and thermal treatment determine the overall porosity and crystallinity. Sol-gel chemistry typically results in large monoliths; however, by utilizing the Stober process or incorporating hard templates (e.g., preformed anodic aluminum oxide and the like) or soft templates (e.g., surfactants, micelles/emulsions, and the like), discrete particles of varying sizes can be generated.

Alternatively, aerosol processes, such as spray pyrolysis or spray drying have also been utilized as methods to generate porous silica particles. For example, silica colloids can be suspended in a solution and sprayed into a thermal source to form particles consisting of the agglomerated/sintered colloids; templates (e.g., polystyrene beads) can be incorporated in the precursor solution, followed by removal via heat or chemical treatment to produce a porous architecture. Sol-gel polymerization chemistry can likewise be incorporated into aerosol methods, using similar templating approaches. In aerosol and sol-gel aerosol methods, the final size of the particle is limited by the ability to nebulize the precursor solution as well as the size of the generated spray droplet.

The chromatographic performance of porous silica particles is intrinsically linked to their shape, size and porosity, as has been extensively described in the scientific literature. Pertinent to the invention described herein is the fact that both the size of silica particles and the nature of their porosity are factors that influence the surface area of the resulting material. In particular, the nature of the porosity determines the accessibility of intraparticle surface area for interaction with the molecules to be separated. In the case of particulate silica, two general groups of commercially available particles have been developed that address the aforementioned factors. Smaller silica particles have been developed by both sol-gel and aerosol methods with diameters of ˜1.5 μm-5 μm and pore diameters in the range of 5 nm-40 nm. While these particles have the advantage of high surface areas and packing efficiencies in chromatography, they necessitate high pressures, and in the case of large biomolecules such as proteins, peptides, glycoconjugates and nucleic acids, the intraparticle pore accessibility is low, if not completely nonexistent, due to the small size of the pore openings compared to the analytes. Conversely, larger silica particles with diameters of 5 μm-50 μm that have larger pores in the diameter range of 40 nm-400 nm have been manufactured by the sol-gel method. These particles are compatible with lower pressures, but have the drawback of low surface areas (≦about 35 m²/g).

Particles presently commercially available for use as a stationary support have allowed for advancements in the chromatographic separations of small molecules and large biomolecules. However, the development and application of a material that incorporates relatively large interconnected pores about 100 nm diameter) with a small particle size about 3 μm diameter) has heretofore not been realized. Such small-diameter particles would allow for high packing efficiency and an overall higher accessible surface area by providing (the advantages of) a high outer surface area as well as a high intraparticle surface area available for interaction with large biological molecules such as proteins, peptides, nucleic acids, and glycoconjugates (e.g., polysaccharides or proteoglycans).

### SUMMARY

According to the present invention, macroporous silica and its use as a stationary phase in liquid chromatography are described.

In one illustrative embodiment, a novel silica particle synthesized by spray pyrolysis (also called spray drying) is described. The application of this type of particle offers distinct advantages over current technology, based on the unique combination of particle size and porosity afforded by the synthesis approach.

In another illustrative embodiment, described herein is the novel application of silica particles synthesized by spray pyrolysis (as described herein) as the support material for the stationary phase packed into an affinity chromatography column. Compared to affinity chromatography columns fabricated using commercially available silica, an affinity chromatography column packed with the novel silica particles described herein offers between a 10-fold and 100-fold increase in binding capacity.

Additional advantages and features of the present invention will become apparent to those skilled in the art upon consideration of the following detailed description exemplifying the best mode of carrying out the invention as presently perceived.

### BRIEF DESCRIPTION OF THE DRAWINGS

The detailed description particularly refers to the accompanying Figures in which:

FIG. 1 shows a scanning electron micrograph (A) and transmission electron micrograph (B) images of silica particles synthesized by spray pyrolysis/drying using salts as a pore template;

FIG. 2 shows (A) a magnified image of silica particles synthesized by spray pyrolysis/drying using salts as a pore template showing pore diameter sizes and (B) the corresponding N₂ adsorption-desorption isotherms of the porous silica particles (FIG. 2(B) Inset: Pore size distributions obtained from BJH analysis of the same samples; surface areas based on 3-point BET analysis are also denoted); and

FIG. 3 shows A) a chromatogram of affinity binding and elution of HRP on a Con A-silica column, B) binding capacity of the Con A-silica column for HRP, C) a chromatogram of affinity binding and elution of AGP on an AAL-silica column, and D) binding capacity of the AAL-silica column for AGP; the arrows in A) and C) indicate the time at which elution buffer was applied.

### DETAILED DESCRIPTION

While the invention is susceptible to various modifications and alternative forms, for the purposes of promoting an understanding of the principles of the invention, reference will now be made to a number of illustrative embodiments illustrated in the Figures and specific language will be used to describe the same. It should be understood, however, that there is no intent to limit the invention to the particular forms described, but rather, on the contrary, the intention is to cover all modifications, equivalents, and alternatives falling within the spirit and scope of the invention.

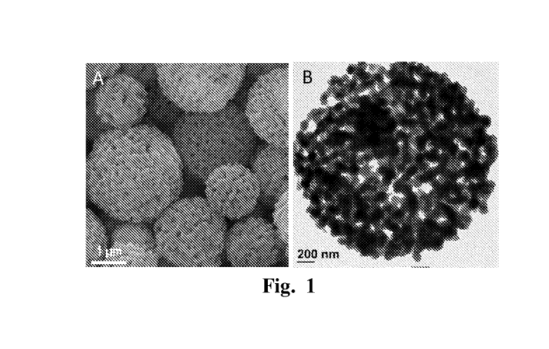

In accordance with one illustrative embodiment of the present invention, a novel silica particle synthesized by spray pyrolysis (also called spray drying) is described. The application of this type of particle offers distinct advantages over current technology, based on the unique combination of the particle size and porosity afforded by the synthesis approach. A scanning electron micrograph (SEM) image of the material, depicting the external surface as well as the intraparticle pore openings, is shown in FIG. 1A. The internal structure of a representative particle is illustrated by the transmission electron micrograph (TEM) image in FIG. 1B. The TEM image confirms the extensive interconnected void spaces inside the particle. The novel silica particle (material) is synthesized by spray pyrolysis of silica colloids, with porosity introduced into the particles by means of salts acting as pore templates (see, Peterson, A. K.; Morgan, D. G.; Skrabalak, S. E., Aerosol synthesis of porous particles using simple salts as a pore template. Langmuir 2010, 26, (11), 8804-8809, the disclosure of which is incorporated herein by reference in its entirety). Aerosol syntheses are ideal for generating particles with diameters≦2.5 μm. The precursor solution consists of silica colloids with diameters ≦100 nm and salts dispersed in a water solution. This solution is then nebulized and carried to a heating element where the silica undergoes hydrolysis and the salt inhibits complete solidification of the colloids. Washing of the product post-synthesis removes the salt, revealing the porous particles.

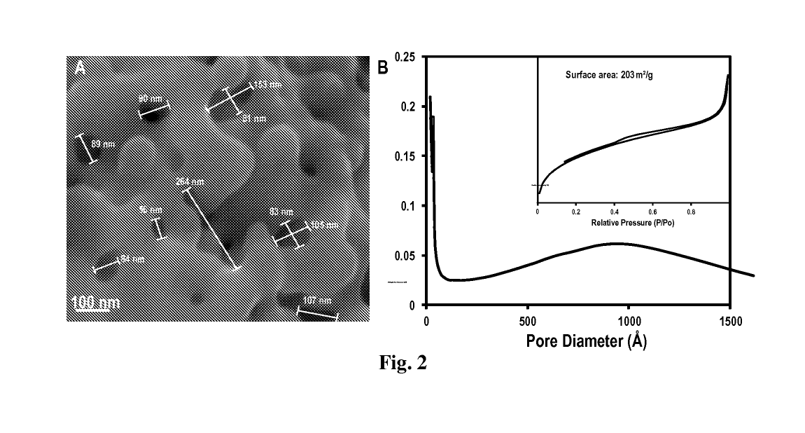

Silica particles synthesized as described above are spherical with diameters up to about 2 μm and pore diameters as large as about 300 nm. The small particle diameters combined with the large interconnected pores provide a support with a high surface area, between 150 m²/g and 300 m²/g, which is accessible to small molecules as well as larger biomolecules (FIG. 2). The application of silica particles synthesized by spray pyrolysis with salts as a pore template to chromatography allows for a distinct combination of advantages over current state-of-the-art silica particles. The particles synthesized by spray pyrolysis offer a high packing efficiency as a result of their small diameters; they have a high surface area that is accessible to small molecules as well as larger biomolecules such as proteins, peptides, nucleic acids, and carbohydrates; and the extensive interconnection of the macropores within the particles facilitates a more rapid mass transfer through the particles than may be achieved with traditional, completely porous particles.

In accordance with another illustrative embodiment of the present invention is the novel application of silica particles synthesized by spray pyrolysis (as described herein) as the support material for the stationary phase packed into a chromatography column. As described in the following Examples section in more detail, the application of the novel silica particles in a technique called affinity chromatography has been demonstrated. Affinity chromatography is a method in which the stationary phase interacts strongly with target chemical/biological molecules in a sample mixture to the exclusion of other moieties, thus allowing for the molecules with the target moieties to be temporarily attached to the stationary phase while the rest of the compounds in the mixture are washed away by the mobile phase. Next, the elution solvent, typically a mobile phase that disrupts the affinity interaction between the stationary phase and the target moieties, is applied, thereby allowing for the molecules containing the target moieties to be eluted from the stationary phase separately from the unbound molecules in the original mixture.

Because of the unusual and extensive porosity of the silica particles described herein, these particles provide a much larger surface area for interaction with molecules in the mobile phase. This property is illustrated by the significant increase in binding capacity shown for an affinity chromatography experiment. Compared to affinity chromatography columns fabricated using commercially available silica, the affinity chromatography column packed with the new silica particles described herein offers between a 10-fold and 100-fold increase in binding capacity.

The presently described technology is illustrated by the following illustrative examples, which are not to be construed as limiting the invention or the scope of the specific compositions and methods described herein.

### EXAMPLES

The present invention was illustratively implemented by functionalizing the novel silica particles described herein with a stationary phase, packing the novel, functionalized silica particles into a chromatography column, and then utilizing the packed column for chromatographic separations. The application was tested by fabricating two affinity chromatography columns utilizing novel silica particles synthesized by spray pyrolysis with salts as a pore template.

Silica particles synthesized by spray pyrolysis with salts as a pore template (as described herein) were functionalized for affinity chromatography with two different proteins, concanavalin A (Con A) and avidin. Con A has a relatively weak affinity for carbohydrates containing a-linked mannose residues (K_{d}˜10⁻⁷ M) as described in the literature. Avidin has an extremely strong affinity (Kd˜10-15 M) for the small molecule, biotin. In fact, the avidin-biotin interaction is too strong for facile release of biotin once it is bound to avidin. Because of this strong binding characteristic, it is common to biotinylate a molecule and then incubate it with an avidinylated support material. In this way, the biotinylated molecule will become anchored to the support via the avidin-biotin interaction.

The two proteins, Con A and avidin, were immobilized on silica particles synthesized by spray pyrolysis with salts as a pore template using a previously described procedure (see, Larsson, P., Glad, M., Lennart, H., Mansson, M., Ohlson, S., Mosbach, K., High-Performance Liquid Affinity Chromatography. In Advances in Chromatography, 1 ed.; Giddings, J. C., Grushka, E., Cazes, J., Brown, P. R., Ed. Marcel Dekker, Inc.: New York, 1983; Vol. 21, pp 41-85, the disclosure of which is incorporated herein by reference in its entirety). Briefly, the silica particles were first coated with 3-glycidoxypropyltrimethoxysilane in toluene with a catalytic amount of triethylamine. The coating reaction was allowed to proceed for 16 hours at 105° C. under reflux conditions. The silica particles were then washed extensively with toluene, acetone, and ether and dried in a vacuum. Next, the epoxy groups on the particles were oxidized to diols in 10 mM HCl at 90° C. with gentle mixing. The particles were then washed with water, ethanol, and ether and dried in a vacuum. The diols were further oxidized to aldehydes with sodium periodate in 90% acetic acid in water by volume. The reaction was performed at room temperature with gentle mixing. The particles were then washed extensively with water, ethanol, and ether and dried in a vacuum. Con A was solubilized in a 20 mM phosphate buffer, pH 7.4, and mixed with the aldehyde-modified silica. The Con A and silica slurry was sonicated for 5 minutes. An aliquot of sodium cyanoborohydride was added to the slurry and the reaction mixture was mixed end-over-end for 48 h at 4° C. During this time, the primary amines on the Con A were covalently linked to the aldehydes, and a Schiff base was formed. The sodium cyanoborohydride was used to reduce the Schiff base to a secondary amine. Avidin was immobilized on the silica particles synthesized by spray pyrolysis with salts as a pore template in the same way, except that a bicarbonate buffer, pH 8.6, was used during the coupling reaction rather than a phosphate buffer. Following covalent attachment of avidin to the support material, an aliquot of biotinylated Aleuria aurantia lectin (AAL) was added, and the mixture was rotated end-over-end for 1.5 h to create a stationary phase of avidin-biotinylated AAL (avidin-bAAL). AAL has an affinity for fucose-containing carbohydrates, as described in the literature.

An affinity chromatography column was packed with the Con A silica and a second affinity chromatography column was packed with the avidin-bAAL silica. The columns were packed using an Akta Purifier fast protein liquid chromatography (FPLC) pump. Briefly, a packing reservoir was first filled with either Con A silica or avidin-bAAL silica in binding buffer (50/50 slurry, v/v), then connected in-line with the liquid pump. The empty column was connected downstream from the reservoir with the end farthest from the reservoir end-capped by a 0.2-μm stainless steel frit. A flow rate of 60 μL/min was used to pack each of the columns with one of the two modified support materials until the pressure stabilized, indicating that the packing process was complete. Following packing, the other end was also end-capped with a 0.2-μm frit. Each column had a 1-mm inner diameter and a 5-cm length. The efficacy of each column to retain molecules exhibiting the specific carbohydrate moieties was tested using standard proteins that have the requisite carbohydrates on them; horseradish peroxidase (HRP) was used to test the Con A column, and α-1-acid glycoprotein (AGP) was used to test the avidin-bAAL column. A dynamic binding curve was generated to demonstrate the binding capacity of the affinity columns.

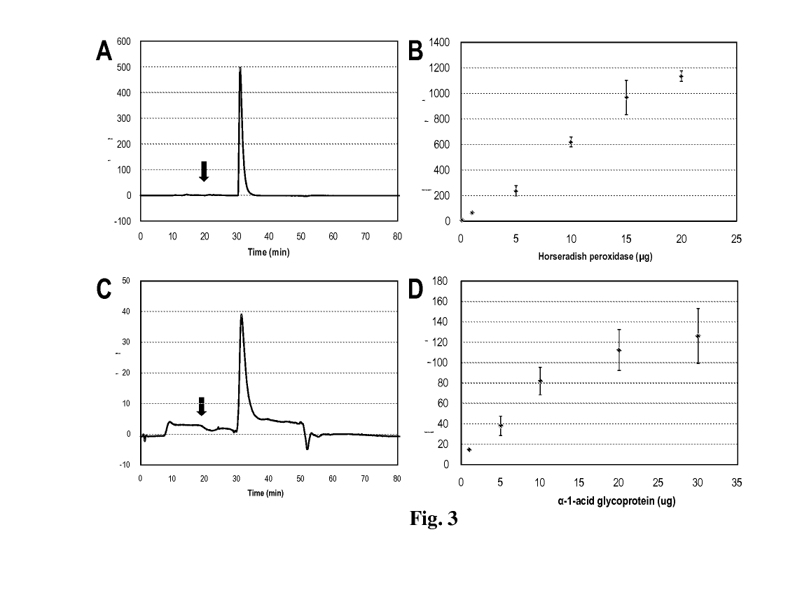

Con A buffers used were as follows: binding—10 mM acetate, pH 5.3; elution—100 mM methyl α-D-mannopyranoside in binding buffer. AAL buffers used were as follows: binding—20 mM phosphate, pH 8.6; elution—100 mM L-fucose in binding buffer. For all affinity chromatography experiments, analytes were injected in 100-μL of binding buffer. Linear velocity was 1.2 cm/min for binding and 2.5 cm/min for elution. The results (FIG. 3) demonstrate that the Con A column was able to bind between 15 μg and 20 μg of HRP injected in a 100-μL aliquot under the chosen experimental conditions, and the AAL column was able to bind a similar amount of AGP. Additionally, it is notable that the biotin-avidin system that was employed to immobilize AAL on the support material could conceivably be used to immobilize any other protein that was first biotinylated, suggesting an extremely broad applicability to the field of affinity chromatography.

Both proteins were retained on the appropriate affinity columns until the elution solvent was applied, at which time they were released from the stationary phase and eluted from the columns (FIG. 3A and FIG. 3C). A protein that does not include/display the appropriate target carbohydrates, namely bovine serum albumin (BSA), was loaded onto the affinity columns as a negative control, and it was observed to pass through the column with the loading buffer before the application of any eluting solvent. Further tests have been performed to characterize the binding capacity of the affinity columns, and they have also been used to specifically extract molecules with the target carbohydrate moieties from human blood serum.

The present invention has been tested with an affinity chromatography system to demonstrate that it provides a unique support material for the stationary phase in a chromatographic column. In the model systems tested, both the stationary phase moieties and the molecules in the mobile phase that were retained were proteins (i.e., large biomolecules, ca. 1 nm-100 nm). The support was thus demonstrated to be suitable for facilitating the interaction between a bulky biological stationary phase moiety and a biological sample molecule. While the material is suitable for affinity chromatography of large biomolecules, there might have been a concern that in more traditional, high-resolution chromatographic separation techniques such as reversed-phase, hydrophilic interaction, normal phase, and hydrophobic interaction chromatography, the resistance to mass transfer of analytes that are similar in size to the pore diameters will contribute adversely to band broadening, and thus detract from the overall separating power of the column. However, a recent publication has demonstrated experimentally that, for analytes that are small relative to the mean pore diameter, there is no significant interaction between the support material and the analytes (see, Wernert, V.; Bouchet, R.; Denoyel, R., Influence of molecule size on its transport properties through a porous medium. Anal Chem 2010, 82, (7), 2668-79). Thus, the increased accessible surface area of the novel particulate silica support material described herein may provide an overall improvement in the separating power of a column by increasing the number of adsorption-desorption events between the analytes and the stationary phase. The pores in the novel particulate silica material described herein are about 50 nm to about 300 nm in diameter; accordingly, the material can be utilized as a support for high-resolution chromatographic separations of analytes that are more than an order of magnitude smaller (e.g., <5 nm).

The invention has now been described in such full, clear, concise and exact terms as to enable any person skilled in the art to which it pertains, to practice the same. It is to be understood that the foregoing describes preferred embodiments and examples of the invention and that modifications may be made therein without departing from the spirit or scope of the invention as set forth in the claims.

We claim:

1. A silica particle having a diameter less than or equal to about 2 μm, wherein the particle is spherical and comprises interconnected pores having a diameter in the range from about 50 nm to about 300 nm.

2. The silica particle of claim 1, wherein the silica particle provides a support with a surface area of about 150 m²/g to about 300 m²/g.

3. The silica particle of claim 1, wherein the silica particle is functionalized with a stationary phase.

4. The silica particle of claim 3, wherein the stationary phase is selected from the group consisting of proteins, peptides, nucleic acids, polysaccharides, and proteoglycans.

5. The silica particle of claim 4, wherein the proteins are concanavalin A or avidin.

6. The silica particle of claim 1, wherein the silica particle is synthesized by spray pyrolysis of silica colloids.

7. The silica particle of claim 6, wherein porosity is introduced into the particle by means of an inorganic salt acting as a pore template.

8. The silica particle of claim 7, wherein the inorganic salt is selected from the group consisting of NaCl, KCl, LiCl, NaNO₃, LiNO₃, combinations thereof, and mixtures thereof.

9.-19. (canceled)

20. A composition comprising a silica particle having a diameter less than or equal to about 2 μm, wherein the particle is spherical and comprises interconnected pores having a diameter in the range from about 50 nm to about 300 nm.

21. The composition of claim 20, wherein the composition is packing material for a chromatography column.

22. The composition of claim 20, wherein the silica particle is functionalized with a stationary phase.

23. The composition of claim 22, wherein the composition is packing material for a chromatography column.

24. A liquid chromatography method, said method comprising the step of contacting a composition comprising a silica particle having a diameter less than or equal to about 2 μm with a target molecule in a sample mixture,
wherein the silica particle is spherical and comprises interconnected pores having a diameter in the range from about 50 nm to about 300 nm, and
wherein the composition is packing material for a chromatography column

25. The liquid chromatography method of claim 24, wherein the silica particle is functionalized with a stationary phase.

26. The liquid chromatography method of claim 25, wherein the method is affinity chromatography.

27. The liquid chromatography method of claim 26, wherein the chromatography column is an affinity chromatography column

28. The liquid chromatography method of claim 27, wherein the affinity chromatography column has between a 10-fold and 100-fold increase in binding capacity compared to an affinity chromatography column fabricated using commercially available silica.

29. The liquid chromatography method of claim 28, wherein the affinity chromatography column has a 10-fold increase in binding capacity compared to an affinity chromatography column fabricated using commercially available silica.

30. The liquid chromatography method of claim 28, wherein the affinity chromatography column has a 100-fold increase in binding capacity compared to an affinity chromatography column fabricated using commercially available silica.
